(19) United States
(12) Patent Application Publication
(10) Pub. No.: US 2016/0022144 A1
(43) Pub. Date: Jan. 28, 2016

(54) SYSTEM AND METHOD OF INTEGRATING PARTICIPANT BIOMETRICS WITHIN AN EVENT TIMING SYSTEM

(71) Applicant: INNOVATIVE TIMING SYSTEMS LLC, St. Louis, MO (US)

(72) Inventor: Kurt S. Hansen, Chesterfield, MO (US)

(21) Appl. No.: 14/776,684

(22) PCT Filed: Mar. 16, 2014
(86) PCT No.: PCT/US14/30107

§ 371 Date: Sep. 14, 2015

Related U.S. Application Data
(60) Provisional application No. 61/791,455, filed on Mar. 15, 2013.

(51) Int. Cl. A61B 5/00; H04Q 9/00

(52) U.S. Cl. CPC A61B 5/0015; H04Q 9/00; H04Q 2209/47; A61B 2562/08

(57) ABSTRACT

A system and method for collecting biometric data from a participant in an event including a sensor positioned on the participant for sensing a current biometric factor of the participant, the sensor including an interface for communicating the sensed current value of the biometric factor, a biometric data collector communicatively coupled to the sensor for receiving the current sensed biometric factor value as biometric data and further communicatively coupled to an RFID tag, and the RFID tag receive the biometric data from the collector and transmitting the received biometric data to an RFID tag reader responsive to a tag read from the tag reader. The system and method can also include the RFID tag reader and the timing system support for the biometric data communication, analysis and reporting.

### CROSS-REFERENCE TO RELATED APPLICATIONS

This application claims the benefit of U.S. Provisional Application No. 61/791,455, filed on Mar. 15, 2013, the disclosure of which is incorporated herein by reference.

### FIELD

The present disclosure relates to event registration and timing systems and, more specifically, to event timing system integrating a biometric monitoring capability into the RFID timing system for health monitoring and reporting of participants in a timed event.

### BACKGROUND

The statements in this section merely provide background information related to the present disclosure and may not constitute prior art.

When an athlete is training and/or competing in an event, they and the event host as well as trainers and medical personnel are interested in the performance and bio data of the participant. This can be for evaluating performance and training as well as safety of the participants. By monitoring in real time various factors such as heart rate, pulse, blood pressure, core body temp, sweat rate, leg turnover, gate of their run, pace as it relates to overall effort, impact of dehydration on performance, cadence, O2 sensor, etc. these parties can help the participant.

However, current systems for personally monitoring these biometric factors as cumbersome and expensive and therefore are not often used by participants, or event hosts, but are typically confined to laboratory settings or in restricted personal training environments. Furthermore, existing systems are not integrated into event timing systems that enable the event organizer to provide biometric monitoring services to participants during an event as well as during event health monitoring of participants and providing biometric reports and messages to event and participant medical personnel nor providing direct event related biometric and performance data back directly to the event participant.

### SUMMARY

The inventor hereof has succeeded at designing a system and method of use at an event with numerous participants that can monitor participant biometric data in real time during the event, report the monitored biometric data during the event such as via the event timing system timing readers. The measured participant measured biometric data is stored and analyzed and can be used to generate further participant data, reports and/or notification during and after the event. This can include compare the during event biometric data against predefined biometric thresholds and ranges and provide alarms, reports or notifications via a display or communication interface to event medical personnel, the participant or a third party associated with the participant.

According to one aspect, a system for reporting biometric data from a participant in an event includes a sensor, an RFID tag and a biometric data module. The sensor is positioned proximate to a body of the participant for sensing a measured biometric parameter of the participant. The sensor has an interface transmitting the sensed measured biometric parameter. The RFID tag is also associated with the participant and positioned proximate to the body of the participant. The RFID tag has a biometric data communication interface and a tag reader communication interface for transmitting a tag identifier to a remote tag reader responsive to a tag read request therefrom. The biometric data module is communicatively coupled to the sensor for receiving the transmitted measured biometric parameter and has a memory storing the received measured biometric parameter. The module also has an output interface communicatively coupled to the biometric data communication interface of the RFID tag for transmitting the measured biometric parameter thereto. The RFID tag receives the transmitted measured biometric message parameter and transmits the received measured biometric parameter to the RFID tag reader along with the tag identifier responsive to the tag read request from the tag reader.

According to another aspect, a system for reporting biometric data from a participant in an event includes a sensor positioned proximate to a body of the participant for sensing a measured biometric parameter of the participant. The sensor includes an interface transmitting the sensed measured biometric parameter. An RFID tag is also associated with the participant and is positioned proximate to the body of the participant. The RFID tag has a biometric data communication interface and a tag reader communication interface with the latter for transmitting a tag identifier to a remote tag reader responsive to a tag read request therefrom. A biometric data module is communicatively coupled to the sensor for receiving the transmitted measured biometric parameter. The module stores the received measured biometric parameter in a memory of the biometric data module and transmits over an output interface the stored measured biometric parameter. The RFID tag receives the transmitted measured biometric message parameter and transmits the received measured biometric parameter to the RFID tag reader along with the tag identifier responsive to the tag read request from the tag reader.

According to another aspect, a method of reporting biometric data from a participant in an event includes processes of sensing at a sensor positioned proximate to the body of the participant a measured biometric parameter of the participant, and transmitting the sensed measured biometric parameter from the sensor. The method can include receiving at a biometric data module positioned proximate to the body of the participant the transmitted measured biometric parameter from the sensor, storing the received measured biometric parameter in a memory of the biometric data module and transmitting from the RFID tag the tag identifier to a remote tag reader responsive to a tag read request therefrom and transmitting the measured biometric parameter over an output interface of the biometric module in conjunction with the transmitting of the tag identifier from the RFID tag.

Further aspects of the present disclosure will be in part apparent and in part pointed out below. It should be understood that various aspects of the disclosure may be implemented individually or in combination with one another. It should also be understood that the detailed description and drawings, while indicating certain exemplary embodiments, are intended for purposes of illustration only and should not be construed as limiting the scope of the disclosure.

### BRIEF DESCRIPTION OF THE DRAWINGS

FIG. 1 is an illustration of a system and method for obtaining biometric data from a participant during an event according to one exemplary embodiment.

FIG. 2 is an illustration of an event timing system with an integrated biometric data collection system using two sets of spaced apart RFID tag readers according to an exemplary embodiment.

FIG. 3 is a block diagram of yet another even timing system with integrated biometric data collection system with an event timing system reflecting additional system components according to another exemplary embodiment.

FIG. 4 is an diagram of a plurality of tag readers positioned along an event route collecting the RFID tag read data as well as the biometric data for each of the tag reads and providing the tag read data and the biometric data to the timing system that in turn provides the participant identification and biometric data to the biometric system according to an exemplary embodiment.

FIG. 5 is a timing diagram of the communication between a tag reader and integrated biometric RFID tag according to one exemplary embodiment.

FIG. 6 is a listing of communication messages and formats that are suitable for use by the disclosed system and method. FIGS. 10A and 10B are an RFID tag case and tag components integrating a wireless interface for collecting biometric data from biometric sensors according to another exemplary embodiment.

FIG. 7 is a block diagram of a system component illustration of an integrated biometric event participant module having both an RFID tag and a biometric participant module packaged as a single participant component module according to one exemplary embodiment.

FIG. 8 is a flexible RFID tag integrating a wireless interface for collecting biometric data from biometric sensors according to one exemplary embodiment.

FIGS. 9A and 9B are an RFID tag case embodiment with an integrated biometric event participant module utilizing an RFID tag packaged in a common case according to one embodiment.

FIG. 10 is a block diagram of the communication between a separately implemented participant biometric module and an RFID tag of a participant and the communications there between and with system components according to one embodiment.

FIG. 11 is a block diagram of the communication between an implemented integrated participant biometric module and an RFID tag and the communications there between and within system components according to one embodiment.

FIG. 12 includes two screen shots of a biometric reporting display for reporting of the collected participant biometric data.

FIG. 13 is a screen shot of an event route or course with a plurality of RFID tag readers positioned along the route for collecting and reporting of the biometric data of a participant by the timing system or the biometric system.

FIG. 14 is an illustration of a display format for reporting of the biometric data of a participant by the timing system or the biometric system such as via a webpage, a display monitor or a mobile application.

FIG. 15 illustrates an exemplary computer system environment according to one embodiment.

FIG. 16 illustrates an exemplary client-server environment according to yet another embodiment.

It should be understood that throughout the drawings, corresponding reference numerals indicate like or corresponding parts and features.

### DETAILED DESCRIPTION

The following description is merely exemplary in nature and is not intended to limit the present disclosure or the disclosure's applications or uses.

In one embodiment, biometric measurement data is collected from a participant in an event such as a sporting event including a timed sporting event such as a race. The biometric data can be any measurable participant data and can include, but is not limited to, measured heart rate, pulse, blood pressure, core body temp, sweat rate, leg turnover, gate of their run, pace as it relates to overall effort, hydration/dehydration, cadence/beat, and Oxygen using an O2 sensor. The biometric data is collected from one or more sensors located on the participant that are configured for collecting the desired biometric data and transmitted to a biometric measurement collection device that includes a memory. The measured biometric data can be received as analog or digital data from the sensor and in many embodiments will include both analog and digital data from a variety of different biometric sensors each configured for measuring a different participant biometric parameter.

In some embodiments, the collected biometric measurement data that is collected and stored can be integrated with the participant event data such as a participant identification number and/or name, by way of example.

This can include an association or integration with an RFID tag of the participant configured for use in the sporting event. The participant's biometric measurement data can be transmitted to the event timing system via the RFID tag reads of the RFID tag readers and can be provided to an event timing system along with the RFID tag read data for the participant. This can also be transmitted via an integrated alternative communication system as well such as a wireless transmitter and receiver using Wi-Fi, Bluetooth, or Mobile communication during the event such as at a split point, start point or end/finish point. Such can be on a selective or subscription basis per participant.

The timing system receives the participant biometric measurement data, stores it, and can internally processes the biometric data in an associated or internal participant biometric data module, which can be separate or integrated with the event timing system or an interconnected component thereof. The event biometric module receives the participant biometric measurement data either continuously or periodically such as when the participant passes a timing or split point. This participant biometric measurement data can be tracked and analyzed, such as by comparison with predefined ranges and thresholds, that may be specific to the particular participant and determinations can be prepared based thereon. These can also be integrated or associated with event timing system determined times, locations and/or distances, for determination and comparison to predetermined ranges or thresholds for such times, locations and/or distances, which may be of particular value, concern or interest to the participant or a third party associated with the participant such as a trainer, coach, or relative, by ways of example. This can be prepared by the biometric measurement module as participant performance data or participant medical data.

Depending on the determinations or as pre-arranged, a report, an alarm or other communication message can be generated by the event biometric module and as the participant biometric measurement data, as participant performance data, or participant medical data. One or more of these can be sent to the participant or an associated third party during or after the event. In one embodiment, the participant medical data is sent to the participant, an associated third party, or to an event medical system or event medical personnel for monitoring or for taking an action based thereon. For example, the participant biometric measurements can be compared it to predetermined threshold or range and can generate a report, display, alarm or notification based on the biometric data comparisons in the form of participant medical data that may indicate a medical situation that needs attention, such as an abnormally high heart rate, body temperature and/or blood pressure, by way of example.

In one embodiment, a system and method for collecting biometric data from a participant in an event including a sensor positioned on the participant for sensing a current biometric factor of the participant, the sensor including an interface for communicating the sensed current value of the biometric factor, a biometric data collector communicatively coupled to the sensor for receiving the current sensed biometric factor value as biometric data and further communicatively coupled to an RFID tag, and the RFID tag receive the biometric data from the collector and transmitting the received biometric data to an RFID tag reader responsive to a tag read from the tag reader. The system and method can also include the RFID tag reader and the timing system support for the biometric data communication, analysis and reporting as will be further described herein.

In another embodiment, a system for reporting biometric data from a participant in an event includes a sensor, an RFID tag and a biometric data module. The sensor is positioned proximate to a body of the participant for sensing a measured biometric parameter of the participant. The sensor has an interface transmitting the sensed measured biometric parameter. The RFID tag is also associated with the participant and positioned proximate to the body of the participant. The RFID tag has a biometric data communication interface and a tag reader communication interface for transmitting a tag identifier to a remote tag reader responsive to a tag read request therefrom. The biometric data module is communicatively coupled to the sensor for receiving the transmitted measured biometric parameter and has a memory storing the received measured biometric parameter. The module also has an output interface communicatively coupled to the biometric data communication interface of the RFID tag for transmitting the measured biometric parameter thereto. The RFID tag receives the transmitted measured biometric message parameter and transmits the received measured biometric parameter to the RFID tag reader along with the tag identifier responsive to the tag read request from the tag reader.

A tag reader that is associated with a timing system can be used at the event in which the participant is involved with the tag reader transmitting a request to the RFID tag. When the RFID tag receives the request from the tag reader, it transmits the tag identifier and the measured biometric parameter in response thereto. The tag reader receives the tag identifier and the measured biometric parameter as transmitted by the RFID tag and transmits the received tag identifier and the measured biometric parameter over a timing system communication interface to a remote system. This can include a timing system having a first communication interface communicatively coupled to the timing system communication interface of the tag reader. The timing system receives the tag identifier and the measured biometric parameter and stores each. The timing system provides the tag identifier or a participant identifier associated with the tag identifier and the received measured biometric parameter over a second communication interface which can be to an internal or external biometric module. The biometric module receiving the tag identifier and the measured biometric parameter and identifies the participant associated with the tag identifier and the received measured biometric parameter. The module includes stored or predefined biometric parameter values, ranges or thresholds that may be particular to the participant or generally defined for all participants. The received measured biometric parameter is compared to the predefined biometric parameter value and a biometric status of the identified participant is determined as a result of the comparing process.

The biometric module can also have an output interface that can transmit the determined biometric status of the participant to one or more predefined biometric reporting devices. To accomplish this, the biometric module can format the determined biometric status for transmitting over the output interface thereof for displaying the determined biometric status on a visual display along with the identification of the participant. As described herein the biometric module can be a module the timing system, a standalone biometric system, a module of a remote participant management system, and a module of a medical monitoring and reporting system.

The timing system can be configured to determine a time associated with each received measured biometric parameter and the biometric module can be configured to determine the biometric status as a function of the determined time.

The timing system can also identify a geographic location of the participant associated with the received measured biometric parameter and the biometric module can determine the biometric status as a function of the identified location. To accomplish this, a participant location system that identifies the location of the participant can be provided with the timing system or apart therefrom.

The predefined biometric parameter value can be a range of values or a threshold value for the biometric value and the determining of the biometric status by the biometric module can includes identifying the existence of a participant alert condition and can be configured to generate an alert message over an output interface to at least one of the biometric reporting devices including a notification of the identified participant alert condition.

In some embodiments, a participant reporting device can be held or carried or otherwise positioned proximate to the participant during the event. The participant reporting device such as a mobile phone or dedicated device can include an input interface that receives the determined biometric status as transmitted from the biometric module and an output interface that provides an indication to the participate of the received determined biometric status of the participant.

In some embodiments, the system can include a participant image capture system that captures an image of the participant during the event such as when the RFID tag is read and the measured biometric data is sensed and provided. In such cases, the captured image can be associated with the measured biometric parameter of the participant.

As should be understood to those of skill in the art, numerous parameters and different values of the same parameter can be measured over time and at different continuous or periodic times during an event. As such, for each tag read or providing of the measured biometric data, the biometric module can determined a first and second and one value for the measured biometric parameter and each can be compared to each other to track the value of the biometric parameter, to compare the different values to each other and to the predefined biometric parameter value based on location, or times such that the determined biometric status of the identified participant can be customized based on the desired monitoring and reporting requirements of systems connected thereto or as may be desired or required by the event organizers, the participant or otherwise. These can include monitoring one or more biometric parameters of the participant over the time of the event such as heart rate, pulse rate, blood pressure, core body temp, sweat level or rate, skin oxidation, breathing rate, and chemical content of a breath.

In another embodiment, the system for reporting biometric data from a participant in an event includes a sensor positioned proximate to a body of the participant for sensing a measured biometric parameter of the participant. The sensor includes an interface transmitting the sensed measured biometric parameter. An RFID tag is also associated with the participant and is positioned proximate to the body of the participant. The RFID tag has a biometric data communication interface and a tag reader communication interface with the latter for transmitting a tag identifier to a remote tag reader responsive to a tag read request therefrom. A biometric data module is communicatively coupled to the sensor for receiving the transmitted measured biometric parameter. The module stores the received measured biometric parameter in a memory of the biometric data module and transmits over an output interface the stored measured biometric parameter. The RFID tag receives the transmitted measured biometric message parameter and transmits the received measured biometric parameter to the RFID tag reader along with the tag identifier responsive to the tag read request from the tag reader.

The output interface of the biometric module can be a wireless or wired interface and can include any suitable communication interface as described below.

In another embodiment, a method of reporting biometric data from a participant in an event includes processes of sensing at a sensor positioned proximate to the body of the participant a measured biometric parameter of the participant, and transmitting the sensed measured biometric parameter from the sensor. The method can include receiving at a biometric data module positioned proximate to the body of the participant the transmitted measured biometric parameter from the sensor, storing the received measured biometric parameter in a memory of the biometric data module and transmitting from the RFID tag the tag identifier to a remote tag reader responsive to a tag read request therefrom and transmitting the measured biometric parameter over an output interface of the biometric module in conjunction with the transmitting of the tag identifier from the RFID tag.

The method can further include providing the measured biometric parameter data from the biometric data module to the RFID tag prior to the RFID tag transmitting the tag identifier and the measured biometric parameter to a tag reader.

In some embodiments the method can include transmitting from a tag reader a request to the RFID tag, receiving at the RFID tag the request from the tag reader, wherein the transmitting of the tag identifier and the transmitting of the measured biometric parameter are each in response to the received request, the tag reader transmitting the received RFID tag and the received measured biometric parameter to a timing system.

This can also include receiving at a timing system that is communicatively coupled to the tag reader transmitted tag identifier and the transmitted measured biometric parameter, storing each, identifying the participant associated with the tag identifier, comparing the received measured biometric parameter to a predefined biometric parameter value and determining a biometric status of the identified participant as a result of the comparing process.

In some embodiments, the method includes determining a time associated with the received measured biometric parameter and transmitting the determined time with the measured biometric parameter, wherein the determining of the biometric status is a function of the determined time. Embodiments can also include receiving at a biometric module the transmitted tag identifier and the transmitted measured biometric parameter, storing each, identifying the participant associated with the tag identifier, comparing the received measured biometric parameter to a predefined biometric parameter value and determining a biometric status of the identified participant as a result of the comparing process. This can include formatting the determined biometric status for transmitting over the output interface thereof for displaying the determined biometric status on a visual display along with the identification of the participant.

In some embodiments, the method includes identifying a location of the participant associated with the received measured biometric parameter and wherein the determining of the biometric status is a function of the identified location.

The method can also include receiving a set of predefined biometric parameter values that are a range of values or a threshold value for one or more biometric parameters. The method can include determining of the biometric status by the biometric module includes identifying the existence of a participant alert condition, further comprising generating an alert message to at least one of the biometric reporting devices including a notification of the identified participant alert condition.

The method can also include receiving the determined biometric status as transmitted from the biometric module at a participant reporting device positioned proximate to the participant during the event, and providing an indication to the participate of the received determined biometric status of the participant. As noted above, the method can also include capturing an image of the participant during the event and associating the captured image with the measured biometric parameter of the participant.

The RFID tag reader queries the RFID tag read for identification of the RFID tag, receiving the RFID tag identifier and the biometric data from the RFID tag, determining a time associated with the received biometric data and transmitting the tag identifier, the determined time and the biometric data to a timing system.

The timing system has a first communication interface coupled to the tag reader and receiving the tag identifier, the determined time and the biometric data, storing each, and providing the tag identifier or a participant identifier associated with the tag identifier to a biometric system or second communication interface.

The timing system includes the biometric system that receives the identification of the participant based on the tag identifier and the determined time and the biometric data. These can be compared to predefined values for such and a determination as to a current participant status based on the measured biometric data for the determined time as a result of the comparison.

The timing system can include a display locally or remotely positioned for displaying the biometric data along with the participant identification and time. The display can includes a plurality of determined times and biometric data for the participant for a multiple different times during the event. The timing system can also provide an alarm or notification or report of the biometric data and any threshold. This can include a communication link to a remote device including a mobile device such as a smart phone of the participant or another third party based on predefined participant communication profile or parameters therein.

In another embodiment, a system provides for collecting biometric data from a participant in an event including the a sensor and a biometric data collector as described above, further including an RFID tag receives the biometric data from the collector and transmitting the received biometric data to an RFID tag reader responsive to a tag read from the tag reader. The RFID tag reader can query the RFID tag read for identification of the RFID tag, receiving the RFID tag identifier and the biometric data from the RFID tag, determining a time associated with the received biometric data and transmitting the tag identifier, the determined time and the biometric data to a timing system.

In another embodiment, the system includes a timing system having a first communication interface coupled to the tag reader and receiving the tag identifier, the determined time and the biometric data, storing each, and providing the tag identifier or a participant identifier associated with the tag identifier to a biometric system or second communication interface.

In one embodiment, a system and method for use at an event with numerous participants that can monitor, report and provide alarms or other notifications for a larger number if not all of the participants. This can include events such as the iron man competitions as well as small and large marathons. The system and method as provided herein provides for the collection of biometric sensor data as observed and provided by one or more biometric sensors on the participant. The sensors can be wires but are preferably wireless using Wi-Fi or Bluetooth wireless communication from the sensor to an integrated participant RFID-Biometric Tag (RB Tag). The RB tag includes a receiver for receiving the sensor data, storing the data and providing the collected biometric data to the RFID tag. The RFID tag can be a specialized tag or a modified RFID event tag e.g., a modification of an RFID tag or chip that is well-known in the event timing industry. The RFID tag has an interface for receiving the locally provided biometric data (BD) from the various sensors and storing the BD in the memory of the RFID tag. The RFID tag is configured to transmit the BD tag to an RFID tag read during RFID tag reads of the event. The RFID tag reader receives not only the standard RFID tag read data (typically multiple tag reads by each reader for each tag), such as tag identification and a determination of the time of each tag read), but also the biometric data (BD) for that particular tag read as stored in the RFID tag and as is the most recent as sensed from the sensors.

The RFID tag readers receive the tag read data, determine the time, and also receive the BD data. The RFID tag readers (TRS) transmit the tag read data TRD and the biometric data BD to the timing system. The timing system TS stores the BD with the TRD for the participant. The TS can include a biometric data analysis module, system or functionality or can provide this collected BD to a remote system over a communication interface. The TS biometric data analysis system or module BS provides for tracking of the BD data for each participant over time. The BS can include predetermined thresholds for the particular participant or for the event as determined by the event host or medical staff. A trainer or participant can set thresholds or limits for the participant. The BS monitors the BD and compares the collected BD data over time to determine whether the current biometrics are within desired range or whether a threshold or trend is out of range. An alarm or notification can be sent to a medical interface or display or to a remote contact such as a mobile phone or webpage of a trainer, or of the participant via an alarm or text message or the like.

Further, the BS can provide a visual display for the event or for a particular participant and can report in real time or after the event the measured and reported BD of the participant. For instance, a medical staff at an iron man event can monitor the BD of each and every participant at each RFID tag read and can evaluate that over time. The participant or participant's trainer can get real time reporting of the BD or can get an after the event report of all collected BD for post event evaluation.

In some embodiments, the current disclosed system collects real-time or stored data from various devices placed on the body or clothing of the athlete and send that data to a CPU contained on an RFID chip, cell phone, or other data collection device typically worn by the athlete. The information can include data collected at sample rates as fast as thousands of a second, or BD data collected periodically as defined by the application or the user through an application interface. The data collected is stored in a memory buffer on the portable device worn by the athlete. In addition, the BD information can be transmitted to a central collection station or RFID timing station using a variety of technologies such as blue tooth, Wi-Fi, 3G or 4G cellular, RFID signals, or any other communication device capable of sending data from one point to another.

As BD data is collected, it may be processed and computations may occur that can be beneficial to the athlete, coaches, or anyone with an interested in the performance of the athlete. Beneficial data can be transmitted directly to the athlete as they continue to exercise or participate in a physical activity. For example, a runner in a Marathon could be monitored by the system worn on their body. Sensors could be placed on their arms, torso and legs. BD data collected can include turnover on their legs, heart rate, pace, and core body temperature. This BD data can be collected at a split point along the course and forwarded to upcoming split points. When the runner approaches the next split point, data comparison can occur and the athlete could be informed through a blue tooth ear piece if their gate is efficient and their pace is consistent with past training exercises. Another example of biometric sensor might include a leg band that monitors change of measure around the calf muscle. As the athlete is racing, measurements could be taken that could show that the athlete is dehydrated or over hydrated. The same sensor can detect that a muscle is swelling beyond acceptable levels.

The types of devices that could be used to collect BD sensor data might include a micro RFID/Blue Tooth reader, general purpose micro-PIC controller, mobile/cell phone, bicycle computer, of any other computing device capable of communicating over a network or storing data. Feedback to the athlete or coach could be accomplished using audio tones, digital display, voice feedback over a speaker or headphone device, heads-up display on the athlete's sun glasses, or a separate display along the course that is accepting data from the device using RFID or other means.

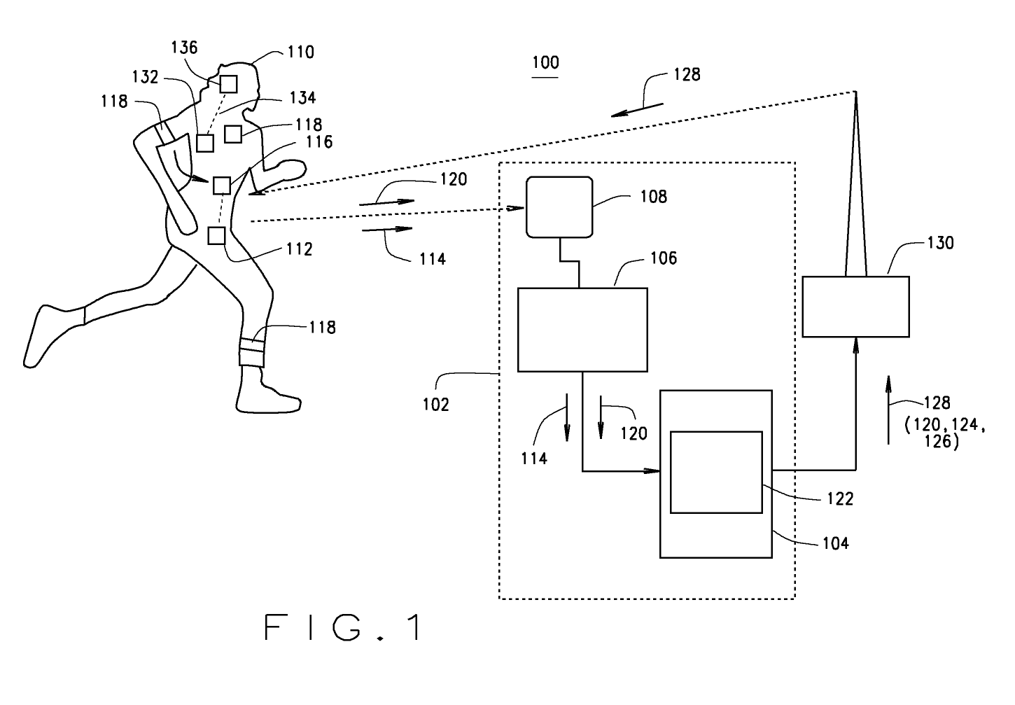

Referring now to the Figures. FIG. 1 illustrates a system 100 that includes an event participant management system 102 utilizing an event timing system 104. The event timing system 104 is coupled to an RFID tag reader 106 having one or more antenna 108. The participant 110 has one or more RFID tags 112 that communicate with the antenna 108 for providing RFID tag reads 114 so that the timing system 104 can uniquely identify the participant 110 based on an RFID tag number of the RFID tag 112 when the participant 110 comes within tag reading proximity of the antenna 108. The participant 110 also includes a biometric data collection and reporting module 116 (referred to as the participant biometric module 116) that can be separate from the RFID tag 112, associated therewith, in communication with or integrated therein. The participant biometric module 116 is communicatively coupled at the participant 110 with one or more participant biometric sensors 118 such as a heart rate sensor, a blood pressure monitor, a sweat/moisture sensor or the like. Each sensor 118 transmits measured biometric data 120 to the participant biometric module 118. The participant biometric module 116 collects the measured biometric data 120 and transmits it directly to the timing system 104 or transmits it or otherwise provides it to the RFID tag 112 that has been modified for receiving the measured biometric data 120. In this embodiment, the RFID tag 112 is configured to not only transmitting the RFID tag number to the antenna 108 during a tag read as tag read data, but also to include the transmission of the measured biometric data 120 with tag read 114. The antenna 108 collects the tag read 114 from the tag 112 that includes the measured biometric data 120 and transmits it via the tag reader 106 to the timing system 104. The timing system 104 can store and process the measured biometric data 120 directly or via a separate or integrated biometric module 122 as described above and herein. The timing system 104 and/or the biometric module 122 transmits the received measured biometric data 120, the participant performance data 124 and/or the participant medical data 126 (hereinafter collecting referred to as the reported biometric data 128) to a biometric reporting communication system 130. The biometric reporting communication system 130 will be described in more detail but is shown in FIG. 1 as being a wireless communication system 130 that transmits the reported biometric data 128 to a predefined biometric reporting system 132, which is shown in FIG. 1 to be a mobile or smart phone 132 of the participant 110. In this example, the smart phone 132 receives the reported biometric data 128 and can be configured to transmit a biometric status message 134 to a participant reporting device 136 such as a blue tooth enabled audio headset or ear piece. In this example, the participant 110 can received direct biometric status messages 134 indicative of the reported biometric data 128 that is based on the measured biometric data 120 during the participants 110 participation in an event.

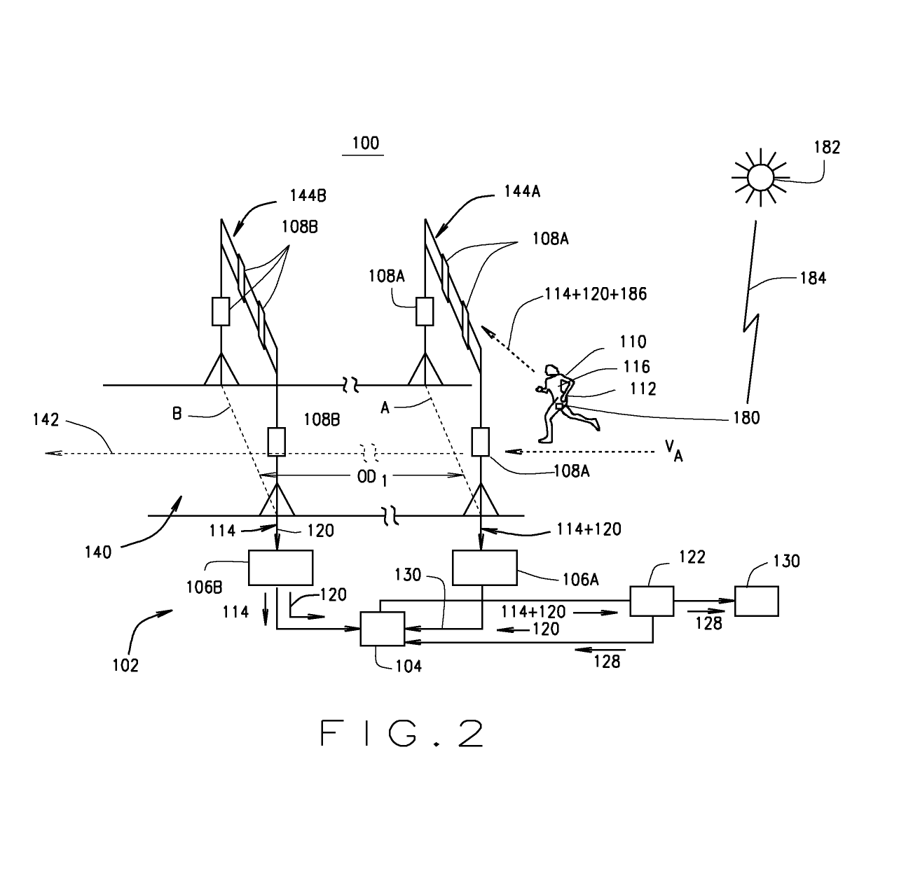

FIG. 2 is a system 100 that includes an event participant management system 102 with an integrated biometric data collection system 122 for a timed running event. This is similar to that shown in FIG. 1, but illustrates participant 110 traveling along event course 140 and participant travel path 142 therein and traveling at a speed of VA. In this illustration, the participant management system 102 includes the event timing 104 with biometric module 122. The timing system 104 is coupled to two tag reader reading systems, a first tag reader system 144A is located along course 140 at point A and includes a plurality of antenna 108A coupled to tag reader 106A. A second tag reader system 144B is located along course 140 at point B that includes antenna 108B coupled to tag reader 106B. Point A is spaced apart from point B at a distance OD1 along course 140. Distance OD1 can be only few feet or can be split points such as miles or kilometers apart along course 140, such as a start line, a split point or a finish line, by ways of example. Each tag read 114 and associated measured biometric data 120 is correlated to the particular point A and point B and such can be provided to biometric module 116 for use in analysis and reporting.

As shown, as the participant with RFID tag 112 and participant biometric module 116 approaches and passes each of points A and B, the first tag reader system 144A will receive both the tag read 114 and the measured biometric data 120 from the RFID tag 112 and/or the participant biometric module 116 and the tag readers 106A, 106B will transmit each tag read 114 and measured biometric data 120 to the timing system 104 via communication link 146. The timing system 104 in this embodiment transmits the participant data and the tag read data to the biometric module 122, which could be a software module within the timing system 104 or a separate system. The biometric module 122 receives the participant identification from the tag read 114 and the measured biometric data 120 and performs the biometric analysis tasks and processes as described herein and then generates the reported biometric data 128 to the biometric reporting communication system 130 for further processing. Further, the biometric module 122 can also transmit the reported biometric data 128 back to the timing system 104 for storage, reporting and other processing of participant data or output to other interfaces and systems.

The timing system 104 in FIG. 2 also includes an optional GPS tracking capability. The participant 110 can include a GPS locator 180 that receives GPS location data 184 from a plurality of GPS transmitters 182 which are usually satellites. The participant location data 186 can also be transmitted to the timing system 104 and the location data 186 can be integrated with or utilized in the analysis of the biometric data 120 by the timing system 104 and/or the biometric module 122.

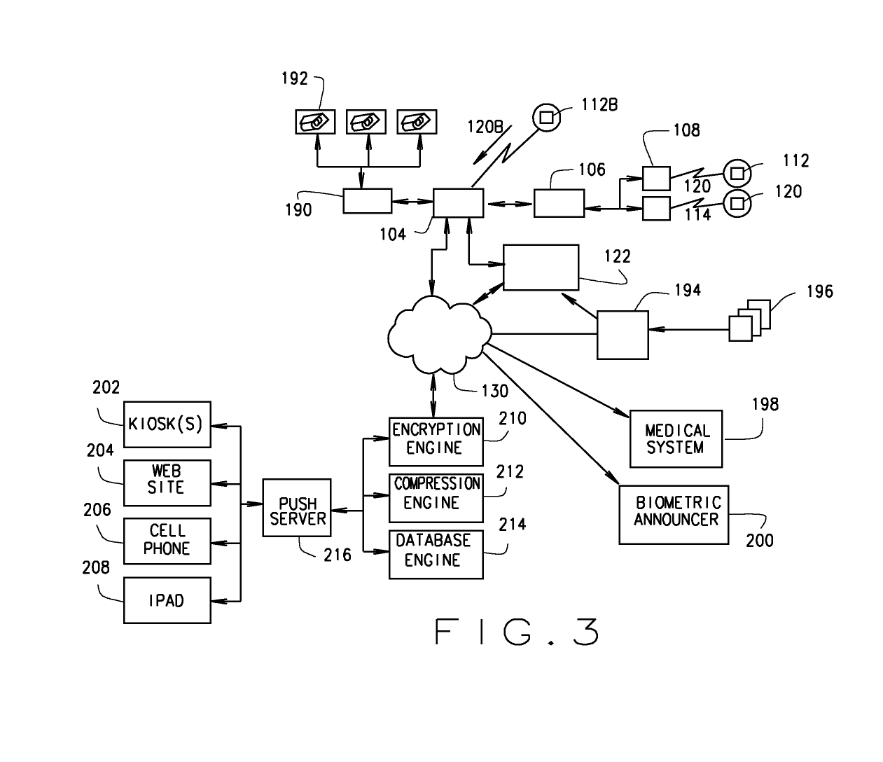

FIG. 3 is a block diagram of an event timing system 102 with integrated participant measured biometric data collection and distribution and reporting system. In this embodiment, the event timing system 104 is communicatively coupled to RFID tag 112 for receiving tag read 114 as well as to participant biometric module 112 for receiving measured biometric data 120 via tag reader 106. However, timing system 104 is also communicatively coupled via a data network such as a wireless network to participant biometric module 112B for receiving measured participant biometric data 120B that is not via the tag reader 106. Further, by way of example, the timing system 104 is coupled to an image capture system 190 that is coupled to one or more image capture devices 192 such as a still camera or a video camera. The images captured by the image capture system 192 are transmitted to the timing system 104 and such images of the participant 110 can be associated with or utilized in the analysis of the biometric data 120 of the participant 110.

Further, a participant registration system 194 can be communicatively coupled to the timing system 104 as well as the biometric module 122. The participant registration system 194 is accessed by a user or participant by using one or more user device 196 for inputting user data as well as defining biometric data for the participant such as ranges and thresholds or other predefined participant biometric data that is utilized by the biometric module during the analysis and comparison with the measured biometric data 120. This can also include the identification or authorization of participant defined outputs and reporting such as defining a telephone number for telephone or message contacting, a url or IP address or a website or user name for a website. For example, a runner participant inputs into system 194 that he wants to be alerted if his core temperature rises above 101 degrees, or if his O2 output falls below 96%. The outputs for the reporting from the biometric module 122 can be provided via network 130 to output system such an event or participant medical system 198, a biometric announcer 200, a kiosk or billboard 202, a web site 204, a mobile or cell phone 206, or another mobile device 208, with any of these being via an application or messaging format or system as may be desired or suitable. The system can include an encryption engine 210, a compression engine 212, a database engine 214 and a biometric data push server 216 in various embodiments and combinations thereof. For example as noted above, if the system detects that the participant's core temperature reaches 102 degrees or that his O2 output reaches 94%, the biometric module 122 will receive this information and compare it to the thresholds received from the registration system 194. The system 122 or timing system 104 can trigger an event or alert including the transmitting of such via one or more participant defined outputs. This can include transmitting an alert to user device 116 that can be a mobile phone 132 that can play an audible, visual or biofeedback alert to device 136 attached to the body or the participant (such as connected to his waistband, his visor or the like) notifying via an audible tone, a visual display or a vibration that the participant in near real time that the predefined thresholds have been exceeded and their alert condition reached.

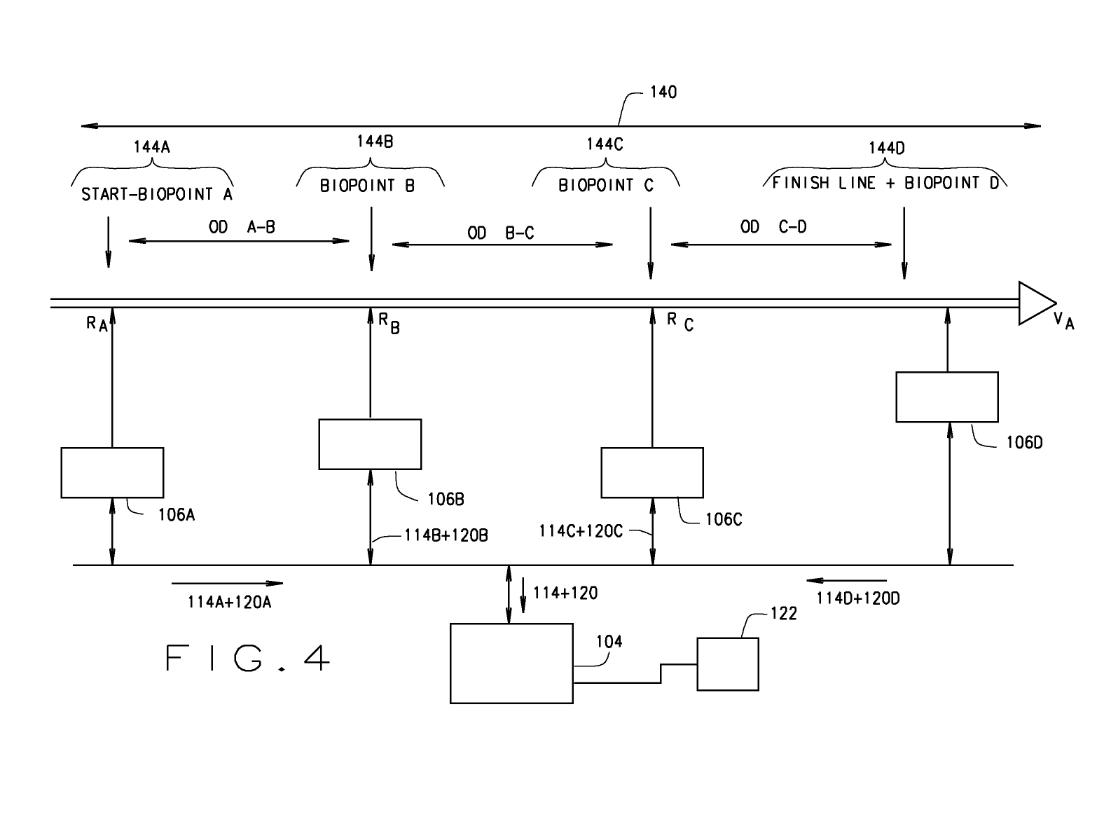

FIG. 4 is an diagram of a plurality of tag readers 106A, 106B, 106C and 106D each positioned along event route 140 at biometric data collection and RFID tag read points A, B, C, and D, respectively. Position A is associated with the start line of the racing event and the initial biopoint A. At the beginning of the event, the participant 110 passes by tag reader 106A and the participants tag is read as 114A and initial start of race measured biometric data 120A is obtained and transmitted to the timing system 104. This process is repeated as the participant 110 passes by biopoint B that is a distance of ODA-B from biopoint A. Tag reader 106B obtains tag read 114B and collects participant measured biometric data 120B and transmits that to the timing system 104. At this time, the timing system 102 or the biometric module can compare the received tag reads 114A and 114B as well as the participant measured biometric data 120A and 120B, and compare these with predefined ranges and thresholds. This process is repeated again as the participant passes biopoint C and tag reader 106C where tag read 114C and participant measured biometric data 120C are collected. This process is completed as the participant 110 passes the finish line located biopoint D and tag reader 106D. The last tag read 114D and last participant measured biometric data 120D is obtained by the timing system 104 and the biometric module 122. In this manner, from the beginning of the event to the end, the participant 110 using the participant biometric module 116 alone or in conjunction with the RFID tags 112 and tag readers 106 automatically provides actual real time or near real time or at least periodic and/or progressive biometric measured data to the biometric module 122 for monitoring, reporting, comparing and analysis. As noted, in some embodiments, one or more of the timing points A, B, C, and/or D and the participant biometric module 116 can provide the measured biometric data 120 alternatively to the timing system 104 or directly to the biometric module 122 via a wireless communication system that is other than the RFID tag reader implementation method as described herein and still be within the scope of the present system and method as that provided in FIG. 4 and otherwise herein.

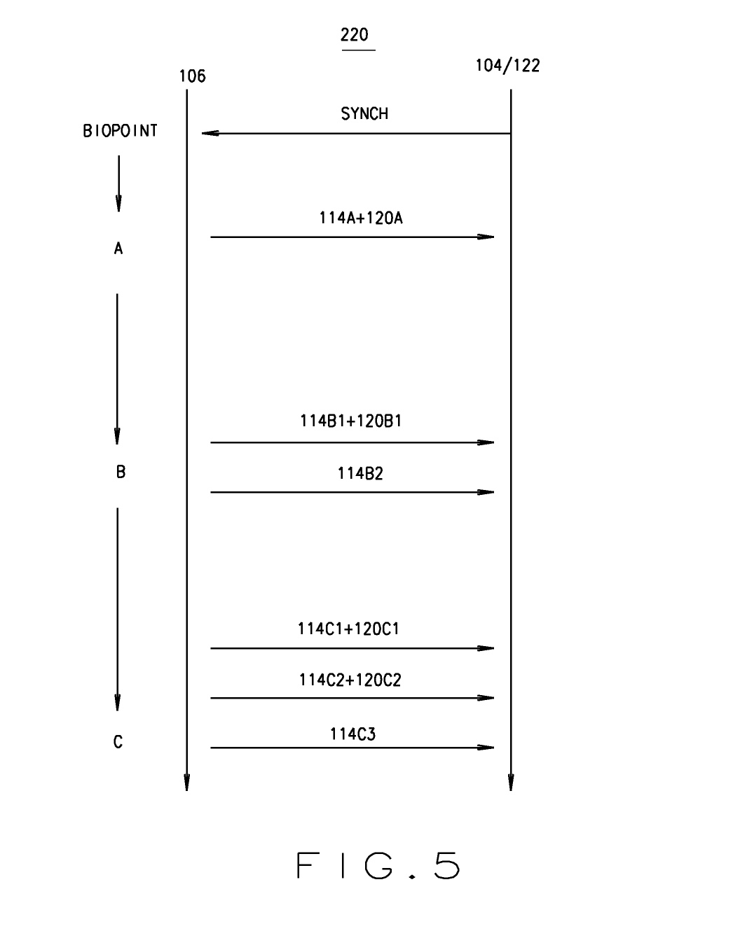

FIG. 5 is a timing diagram 220 of the communication between a tag reader 106 and a coupled timing system 104 and/or biometric module 122. As shown initially the system Synch, then as the participant 110 passes by biopoint A, the tag reader 106A transmits one or more (shown as one for this example) a first tag read 114A and first measured biometric data 120A is transmitted. Next, as the participant passes biopoint C, a first tag read 114B1 and first measured biometric data 120B 1 is transmitted. In this case, the tag reader 106B at biopoint B also sends a second tag read 114B2, but does not send a second measured biometric data as it may not have received a second transmission from the participant biometric module 116 as the data may not have changed. Next as the participant 110 passes biopoint C, a first tag read 114C1 and first biometric data 120C1 is received, but as this is the finish line, a second tag read 114C2 is also obtained as well as a second biometric data 120C2. A third tag read 114C3 can also be received, but may not include a third biometric data read.

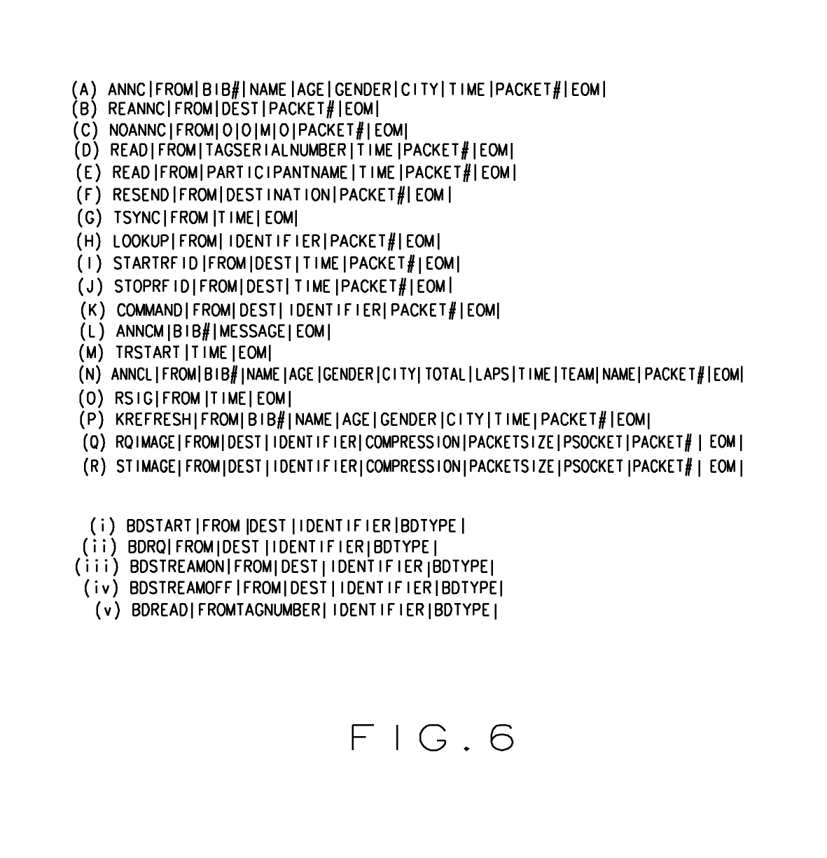

FIG. 6 is a listing of communication messages and formats that are suitable for use by the disclosed system and method. FIGS. 10A and 10B are an RFID tag case and tag components integrating a wireless interface for collecting biometric data from biometric sensors according to another exemplary embodiment. Generally, as known to those of skill in the art, the communication messages and their formats are self-explanatory. In this case, the timing system 104, the tag reader 106, the RFID tag 112, the participant biometric module 116, the biometric module 122 as well as the other system components as described herein such as with regard to FIG. 3 above, but not limited thereto, can communicate over the data network 130 using the messages illustrated in FIG. 6. This can include, but is not limited to, messages BD Start which starts the transmission of collection of the biometric data 120, BDRQ which requests the biometric data 120 to be transmitted from one system to a requesting system, BDSTREAMON and BDSTREAMOFF, which turns on and off the streaming of biometric data 120 from one system to another, and BDREAD that reads the biometric data 120 from a remote system or database. Of course other messages for communicating biometric data 120 within a system 100, a system 102 or among components therein or with and between the timing system 104, is also possible and considered to be within the scope of the present system.

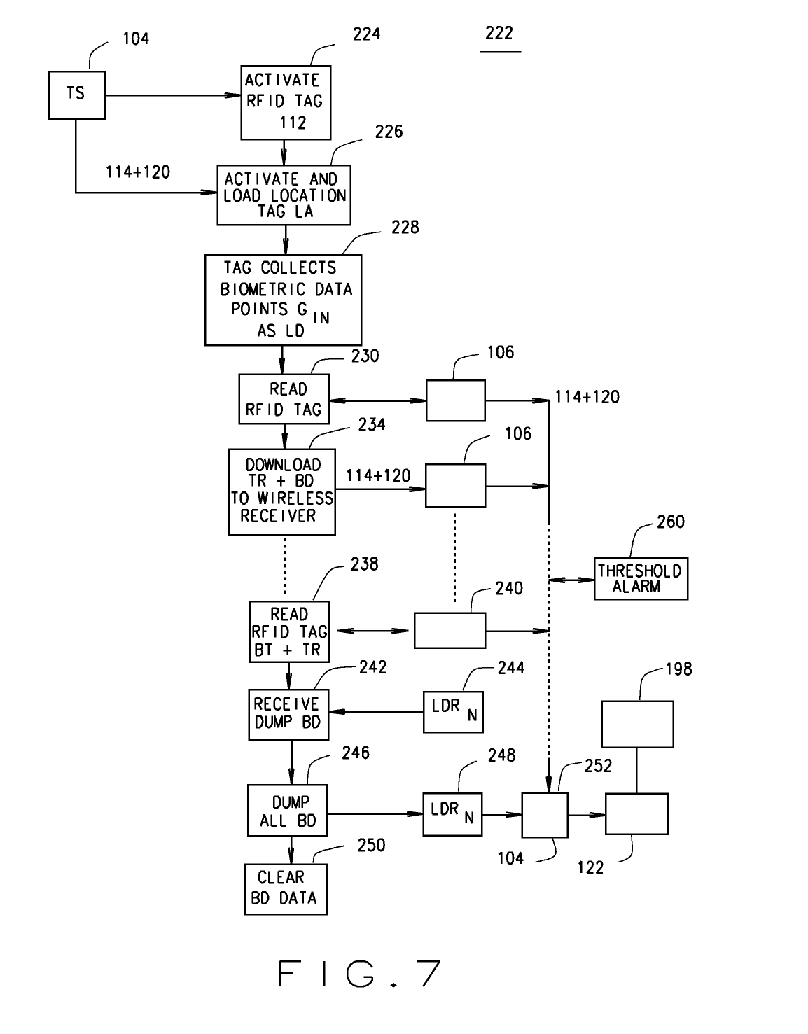

FIG. 7 is a block process flow diagram of the processing of the biometric data 120 as received from an RFID tag reader 106 from a participant 110 during the participant's traversing of a course 140. The timing system 104 initiates an activation of the RFID tag 112 positioned with the participant in process 224 via communication through tag reader 106. The timing system 104 can also provide the RFID tag number or participant name or number and any known biometric data. In this embodiment, the participant also has a location tag or location capability such as a GPS receiver that is activated in process 226. As the participant 110 is involved in the event, the participant measured biometric data 120 is sensed by one or more sensors 118 and collected by the participant biometric module 116 which can also include any known location data from the location module in process 228. When the participant 110 passes a biometric point such as an RFID tag read point having a tag reader 106, the RFID tag 112 is read by the tag reader and a tag read 112 and the measured biometric data 120 is downloaded or obtained by tag reader 106 and provided to the timing system 104 in process 234. This is continued as the participant 110 traverses the course 140 by each biometric point and each tag reader 106. Location data can also be obtained either via the tag reader 106 or otherwise. During this process, the timing system 104 may determine that needs to obtain all current participants biometric data 120 as a data dump that may include multiple biometric points and such request is sent in process 242. This could be also initiated by the location device 244. As a result, all of the collected biometric data 120 can be dumped to the next biometric data receiver (either tag reader 106 or other collector 106B) as in process 246 where it is dumped and transmitted via process 248 to the timing system 104. After the participant biometric data 120 is dumped or otherwise transmitted from the participant biometric data module 116, the module can clear its local memory as in process 250. The timing system 104 communicates the received biometric data 120 to the biometric module 120 that in turn can transmit biometric based data such as reported biometric data 128 to a medical system 198 or another output as shown in FIG. 3. Also, if it is determined after processing that the received measured biometric data 120 deviates from a predefined range or threshold during analysis thereof, a threshold alarm or message or notice or report can be generated in process 260 by one of the means as described in FIG. 3 or otherwise.

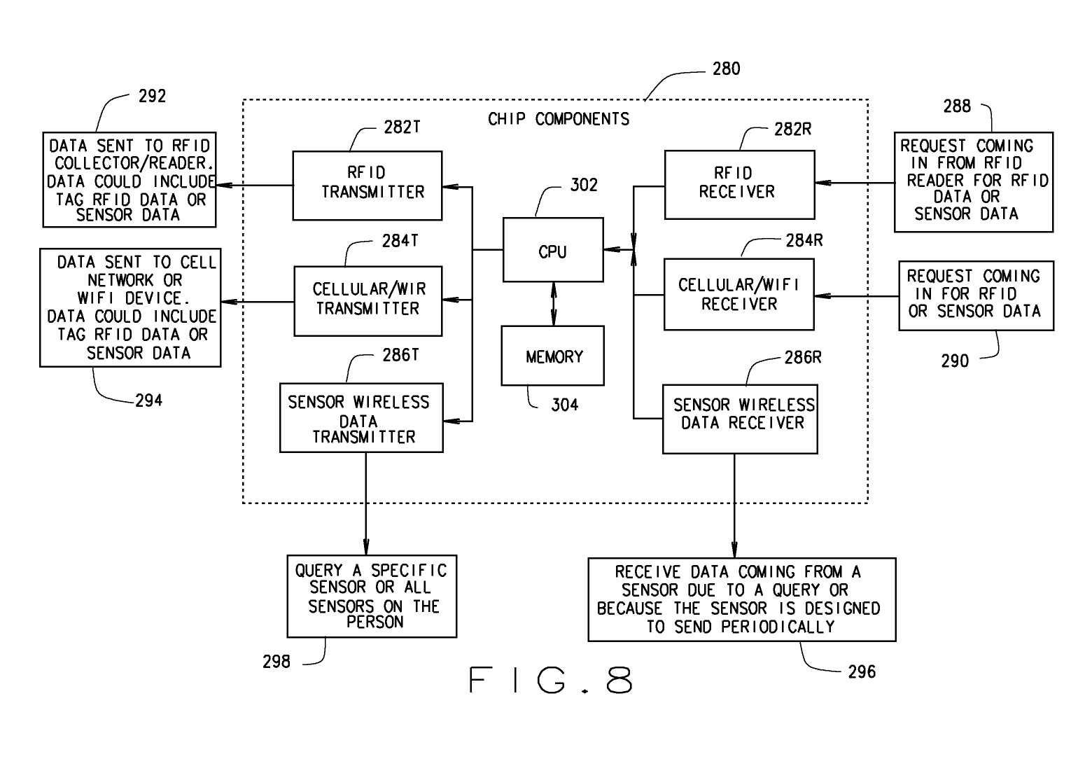

FIG. 8 is a block diagram of is a functional component illustration of an integrated biometric event participant module 280 having both an RFID tag 112 and a biometric participant module 116 packaged as a single participant component module 280 as on example embodiment. As shown, the combined module 280 can have one or more processors or CPU 302 and a memory 304 as is described in more detail below. Further, functionally the module 280 has a RFID receiver 282R that receives a request from an RFID reader 106 for RFID data 112 as well as possibly a separate request for biometric data 120. The module 280 can also have a wireless receiver interface 284R that receives a request for the biometric data from a compatible wireless device 290. The module 280 can also have a local wireless interface 286R for receiving the biometric data 120 from one or more sensors 118. The received biometric data 120 is received by the processor 302 and stored in memory 302. After receiving a request via interface 282R or 284R, the processor 302 can access the stored biometric data 120 from the memory 304 and prepare an appropriately packaged biometric data message 120 that can be transmitted via an RFID transmitter 282T to an RFID tag reader in process 292 or can be transmitted via wireless transmitter 286T to a wireless device in process 294. The module 280 can also be configured to query the sensors 118 to obtain biometric data 120 via wireless sensor transmitter 286T as shown in process 298.

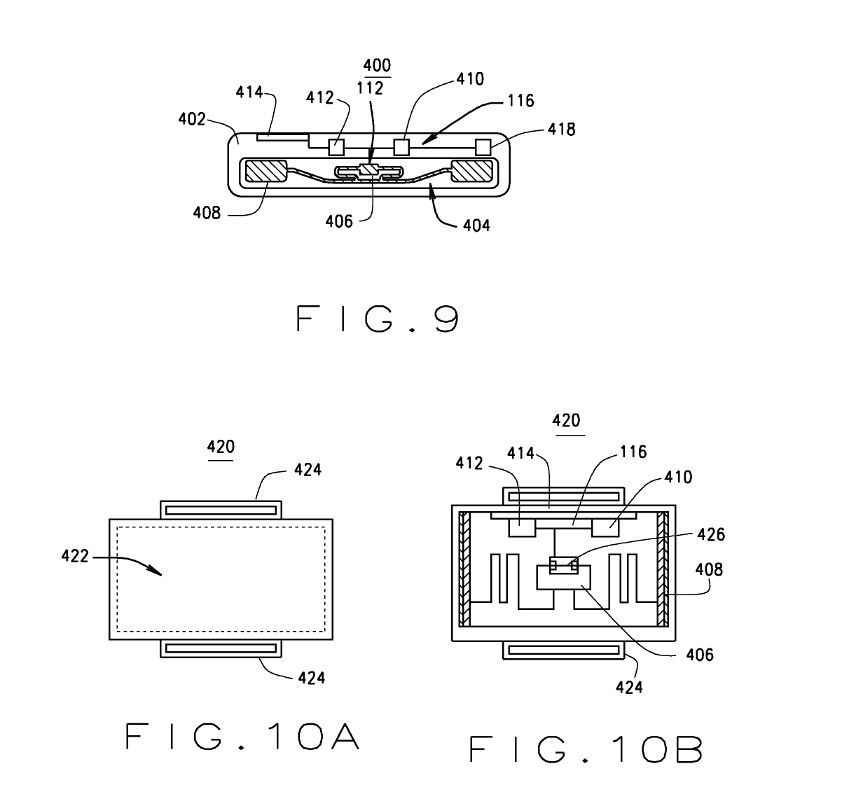

FIG. 9 illustrates one possible physical implementation of an integrated biometric event participant module 150 utilizing a flexible RFID tag 112 integrated with a wireless interface and a participant biometric module 116 for collecting biometric data 120 from participant biometric sensors 118 and transmitting RFID tag reads 114 and the measured biometric data 120 to an antenna 108 and an RFID tag reader 106 associated with a timing system 104 and biometric module 122. In this embodiment of an integrated tag 400, the flexible RFID tag 112 and the participant biometric module 116 are each mounted or manufactured on a common substrate 402 such as Mylar, by way of example. The RFID tag 112 can also have a body 404 on which a RFID tag processor 406 and one or more RFID tag antenna 408 is mounted. In this example, the RFID tag 112 is a passive RFID tag and therefore no power source is provided, however, in other embodiments a power source can be provided for and active RFID tag 112. The participant biometric module 116 is also mounted or manufactured on the substrate 402 wherein a biometric module processor 410 and memory 412 are communicatively coupled and also coupled to a local wireless interface 414 that can include a local antenna such as a Bluetooth or Wi-Fi interface and antenna. As the participant biometric module 116 is constantly monitoring, receiving or requesting biometric data from one or more biometric sensor 118 that is in proximity to the module 150, a biometric module power source 418 is provided. The biometric event participant module 150 can also be referred to as an RFID tag or chip with integrated biometric collection and transmission.

FIGS. 10A and 10B are another embodiment of that shown in FIG. 9 but in this example, the module 420 in configured in a hardened package or case 422. In this example, the case 422 can include one or more case mounts 424 for attaching the module 420 such as by a strap. Inside the RFID tag case 420, the arrangements of the RFID tag 112 and biometric module 116 can be similar to that shown in FIG. 9. In this example, however, the two are interconnected via communication interface 426. In this manner, the RFI tag 112 and biometric module 116 can share data and information for their herein described operations.

FIG. 11 is a block functional diagram of the communication between a separately implemented participant biometric module 116 and an RFID tag 112 of a participant 110 and the communications there between and with timing system 104 according to one embodiment. In this example, the module 280 has a biometric module 116 that receives via the wireless sensor interface 286 biometric data 120 from sensors 118. The biometric module 116 can transfer the biometric data 120 to the RFID tag 112 for transmission via the RFID interface 282 to tag reader 106. In the alternative, such as in a data dump mode, the biometric module 116 can transmit the biometric data 120 via wireless interface 284 to a wireless communication system 130. In either case, the biometric data 120 is transmitted either directly from the tag reader 106 and/or the wireless communication system 130 to the timing system 104 or the wireless communication system 130 and/or the tag reader 106 can transmit the received biometric data 120 to the other that then sends the collected data to the timing system 104.

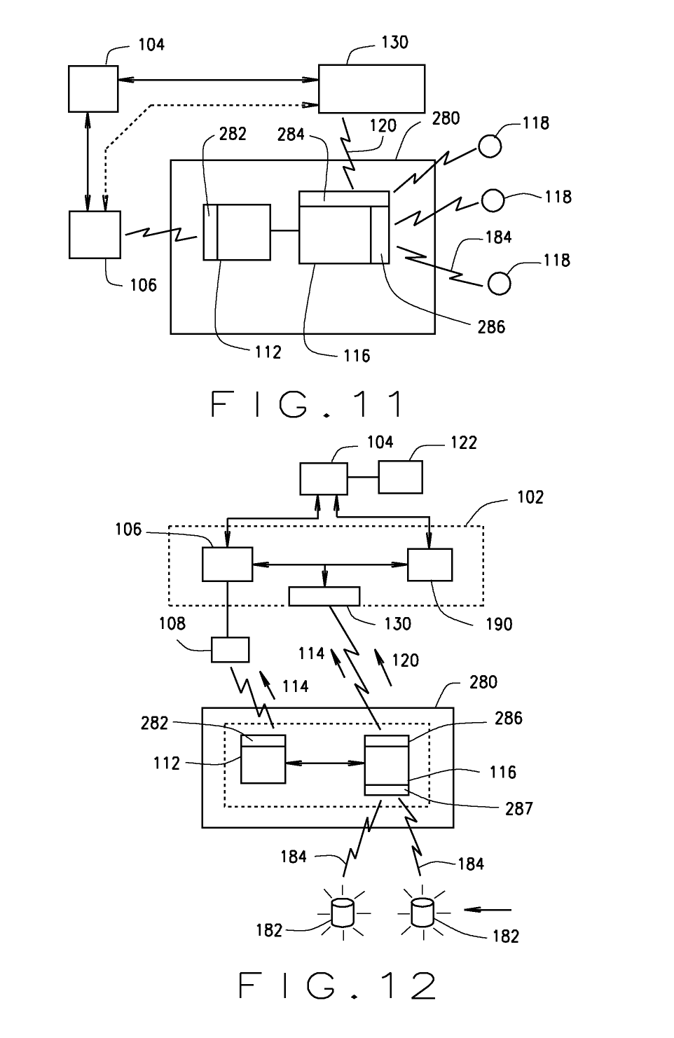

FIG. 12 is similarly a block diagram of the communication between an implemented integrated participant biometric module 116 and an RFID tag 112 and the communications there between and with system components according to one embodiment but in this example, the biometric module 116 is also configured with a location receiver 287 that receives location data 184 from location transmitters 182. The location data 184 can also be transmitted with the biometric data 120 or shared with the RFID tag 112 that can be transmitted with the RFID tag read 114. Also, the tag read 114 and location data 184 can also be separately transmitted via wireless interface 286 to the wireless communication system 130.

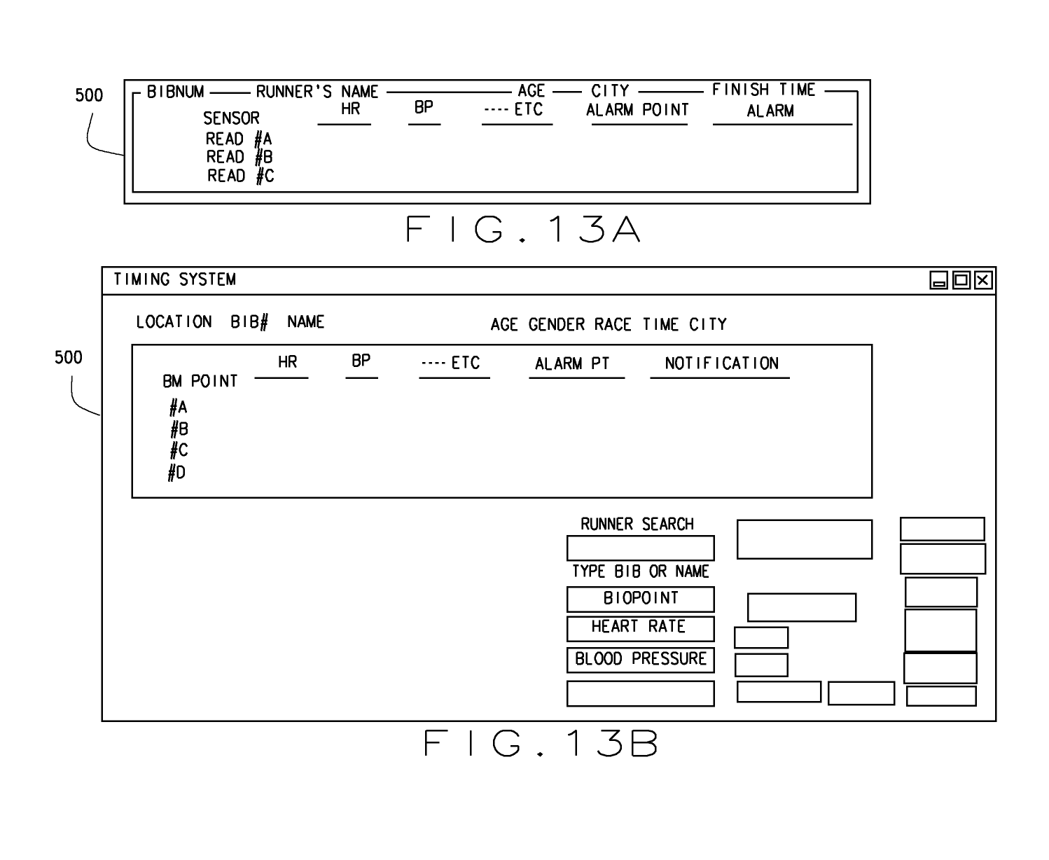

FIG. 13 includes two screen shots 13A and 13B of a biometric reporting display 500 for reporting of the collected participant biometric data. The biometric reporting display 500 can include any type of formatted variations and combinations of the received measured biometric data 120, the participant performance data 124 and/or the participant medical data 126 (hereinafter collecting referred to as the reported biometric data 128). As shown in FIGS. 13A and 13B, this can be on an individual participant basis such as by bib or participant name or number that would include the biopoint at which the biometric data was obtained as well as any of the biometric data or reports or issues identified in the analysis. As shown this can include each biometric point A, B, C, and D, the heart rate (HR), the blood pressure (BP) any potential identified alarm or notification resulting from a comparison of the measured biometric data 120 to predefined ranges or thresholds.

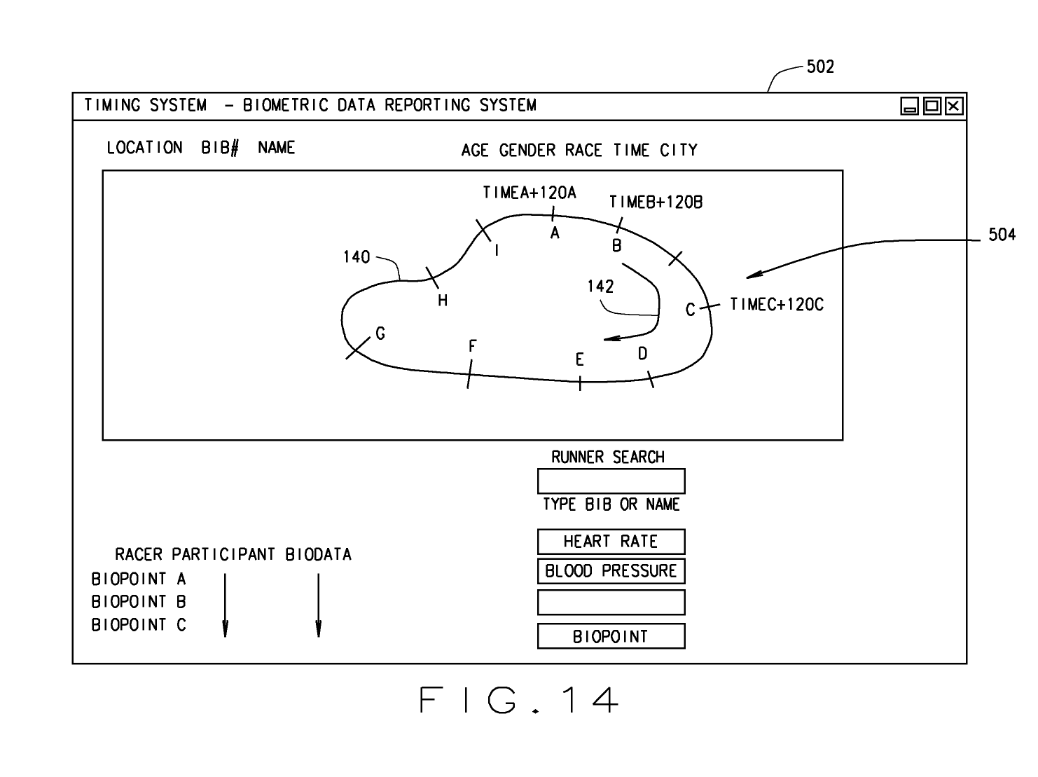

FIG. 14 is a screen shot 502 that is similar but in this embodiment the display 502 includes a mapped illustration of the event route 140 with a visual display of the biopoints A, B, C, etc. In this example, the participant 110 following course 140 has displayed the time and biometric data 120 at biopoint A as Time A and biometric data 120A, at biopoint B as Time B and biometric data 120B, and at biopoint C as Time C and biometric data 120C. In this graphic depiction, a viewer can track the participant 110 about the course 140 and also monitor their biometric data 120 as may be compared to predefined ranges or past experience or medically defined targets or safety levels.

The displays of FIGS. 13A, 13B and 14 can be provided on any suitable output or reporting system such as described by ways of example in FIG. 3 and as discussed above. These can include a display format for reporting of the biometric data of a participant 110 by the timing system 104 or the biometric module 122 such as via a webpage 204, a kiosk display monitor 202 or a mobile application 206.

### Exemplary Digital Processing System Environment

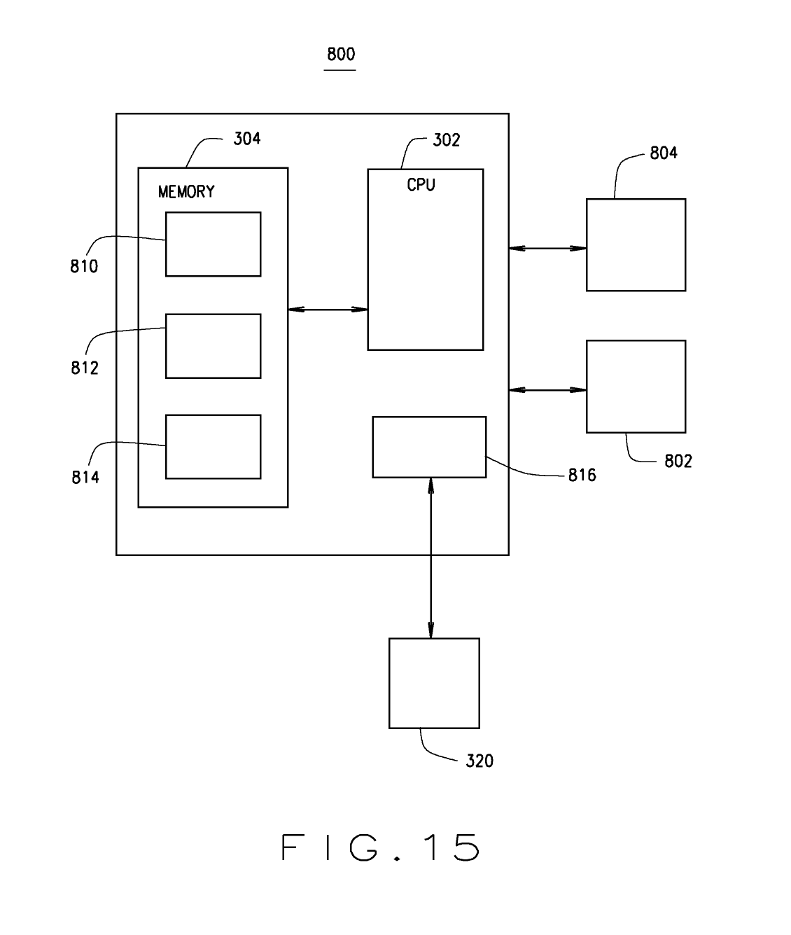

The systems, platforms, servers, applications, modules, programs, and methods described herein for the event participant management system 102 including the timing system 104 and the biometric module 122 among other components. Each of these can include one or more a digital processing systems 800 as shown in FIG. 15. Each component can include one or more hardware central processing units (CPU) 302 that carry out the functions as described above. The digital processing system 800 includes an operating system configured to perform executable instructions for the operation thereof. In most embodiments, the described digital processing systems 800 includes one or more memory devices 304, a display 802, one or more input devices 804, and in some embodiments can include a sound output device such as an alarm or status or verification signal. In some embodiments, the digital processing system 800 can be connected to one or more data networks 320 that can be a wired network, a mobile network, a wireless network such as a Wi-Fi or a Bluetooth™ network or a wired data network. These data networks 320 can be utilize to access the Internet or an intranet such as for accesses to the World Wide Web or other Internet based services. These can include, but are not limited to such data network accessible systems or applications such as a data storage device, a cloud service, an application server, a terminal or exchange server. In some embodiments, the digital processing system 800 is a non-portable device, such as a server or a desktop computer but in many embodiments it can be a portable device, such as a laptop, tablet computer, a mobile telephone device or a digital audio player.

The systems, platforms, servers, programs, and methods disclosed herein for one or more components or features of the system 100, the event management system 102, RFID reader 106, the timing system 104 or the biometric modules 116 and 122 can include one or more computer programs each of which are composed of sequences of computer executable instructions for the digital processing system's CPU each of which are developed to perform one or more specified tasks. Those of skill in the art will recognize that the computer program may be written in various computer programming languages having one or more sequence of instructions. The computer program can be loaded to the CPU 302 or associated memory 304 via a data network connection 320 or a local memory device, but are increasingly via a data network download. Typically, a computer program such as the operating system 810 is loaded by local memory device 304 such as CD or DVD. In some embodiments, the computer program is delivered from one location to one or more locations and can be increasingly distributed via a cloud computing or application service. In various embodiments, the computer program comprises, in part or in whole, one or more web, web browser, mobile, standalone or applications, extensions, add-ins, or add-ons, or combinations thereof. The systems, platforms, servers, programs, and methods disclosed herein above and throughout include, in various embodiments, software, server, and database modules. The software modules are created by techniques known to those of skill in the art using machines, software, and languages known to the art, some of which are disclosed above.

As noted, a digital processing system 800 typically includes one or more memory or data storage devices 304. The memory 304 stores data including the operating system 810 and application programs 812 as well as operating data 814 on a temporary or permanent basis. In some embodiments, the memory 304 can be volatile and requires power to maintain stored information but can also be non-volatile and retains stored information when the digital processing system 800 is not powered. Further, the memory 304 can be located with the digital processing systems 800 or can be attachable thereto either physically or via a data network connection to a remote memory 304. In some embodiments, the memory 304 can also include flash memory devices, solid state memory, magnetic disk drives, magnetic tape drives, optical disk drives, cloud computing systems and services, and the like.

As noted, the digital processing system 800 includes an operating system 810 configured to perform executable instructions which is stored in memory 304. The operating system can include software, including programs and data, which manages the device's hardware and provides services for execution of software applications/modules. Those of skill in the art will recognize that suitable operating systems can include, by way of non-limiting examples, Apple OS®, Microsoft® Windows®, Microsoft®, Windows®, Apple® Mac OS X®, UNIX®, and UNIX-like operating systems such as GNU/Linux®. In some embodiments, the operating system can be provided by cloud computing. Those of skill in the art will also recognize that embodiments of the remote control panel and some components of the primary control panel system may also be implemented using suitable mobile smart phones that include mobile operating systems including, by way of non-limiting examples, Nokia® Symbian®, OS, Apple® iOS®, Research In Motion® BlackBerry OS®, Google® Android®, Microsoft® Windows Phone®, OS, Microsoft® Windows Mobile®, OS, Linux®, and Palm® WebOS®.

The digital processing system 800 can include a visual display 802. In some embodiments, the display 802 can be computer controlled cathode ray tube (CRT) or an optical projector, but is increasingly a flat screen such as a liquid crystal display (LCD), a plasma display, a thin film transistor liquid crystal display (TFT-LCD), a light emitting diode (LED) or an organic light emitting diode (OLED). In other embodiments, the display 802 can also be a combination of devices such as those disclosed herein. Typically they are located proximate to one of the digital processing systems 800 but in some embodiments, the display can be remotely located such as a billboard using LED or electrowetting technology.

The digital processing system 800 can also include one or more an input devices 804 that can be a push button, a key switch, a switch, a keyboard, a touch screen or keypad but these can also include a pointing device such as, by way of non-limiting examples, a mouse, touchpad, light pen, pointing stick, trackball, track pad, joystick, game controller, stylus, multi-touch screen, a microphone that captures voice or other sound inputs or an optical image capture device that can capture images or motion or other visual input. In still further embodiments, the input device 804 can be a combination of devices such as those disclosed herein.

In some embodiments, the digital processing system 800 optionally includes one or more sound output devices (not shown but known to those of skill in the art). These sound output devices can be a set of speakers, a pair of headphones, earphones, or ear buds. The speakers can be of any technology including a flat panel loudspeaker, a ribbon magnetic loudspeaker, an electro-acoustic transducer or loudspeaker or a bending wave loudspeaker, or a piezoelectric speaker. In still further embodiments, the sound output device can be a combination of devices such as those disclosed herein.

Such systems utilize one or more communications networks 320 can include wireline communications capability, wireless communications capability, or a combination of both, at any frequencies, using any type of standard, protocol or technology. In addition, in the present invention, communications network 320 can be a private network (for example, a VPN) or a public network (for example, the Internet). A non-inclusive list of exemplary wireless protocols and technologies used by communications network 320 includes BlueTooth™, general packet radio service (GPRS), cellular digital packet data (CDPD), mobile solutions platform (MSP), multimedia messaging (MMS), wireless application protocol (WAP), code division multiple access (CDMA), short message service (SMS), wireless markup language (WML), handheld device markup language (HDML), binary runtime environment for wireless (BREW), radio access network (RAN), and packet switched core networks (PS-CN). An exemplary non-inclusive list of primarily wireline protocols and technologies used by communications network 320 includes asynchronous transfer mode (ATM), enhanced interior gateway routing protocol (EIGRP), frame relay (FR), high-level data link control (HDLC), Internet control message protocol (ICMP), interior gateway routing protocol (IGRP), internetwork packet exchange (IPX), ISDN, point-to-point protocol (PPP), transmission control protocol/internet protocol (TCP/IP), routing information protocol (RIP) and user datagram protocol (UDP). As skilled persons will recognize, any other known or anticipated wireless or wireline protocols and technologies can be used.

In accordance with the description provided herein, a suitable digital processing system 800 can include, by way of example, server computers, desktop computers, laptop computers, notebook computers, tablet computers, mobile phones such as smart phones, audio devices, personal digital assistants, netbook computers, smartbook computers, subnotebook computers, ultra-mobile PCs, handheld computers, Internet appliances, and video game systems both portable and fixed.

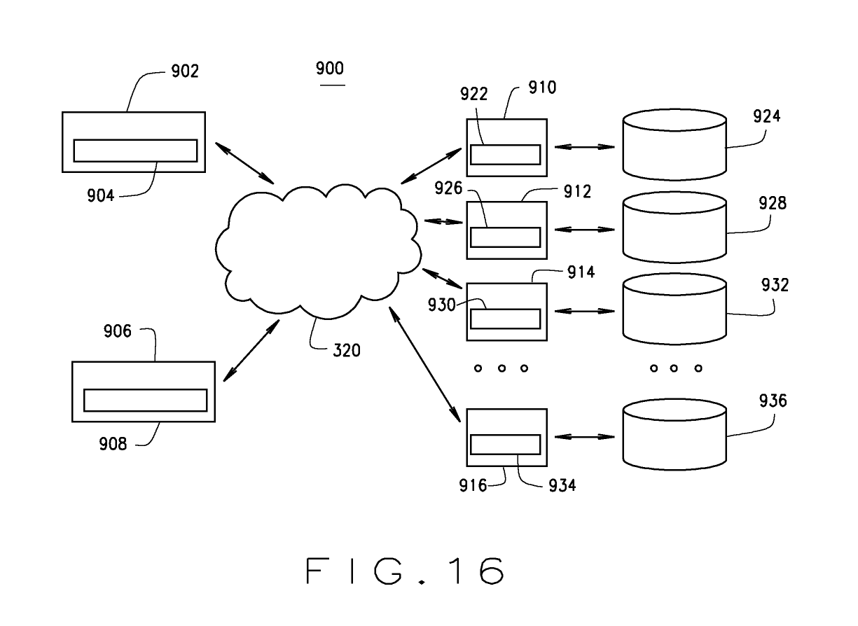

FIG. 16 illustrates a detailed exemplary client-server environment 900. Environment 900 of FIG. 16 includes the aforementioned communications network 320, a plurality of clients 902, 906 and a plurality of servers 910, 912, 914, 916 connected to network 320. The servers 910, 912, 914, 916 are shown connected to a plurality of database servers (DSs). Specifically, server 910 is connected to DS 924, server 912 is connected to DS 928, server 914 is connected to DS 932, and server 916 is connected to DS 936. As one example, the timing system 104 can be implemented as a server 914 and one or more biometric modules 122 can be implemented as a client, 902, 906.

The clients 902, 906 and the servers 910-916 are nodes connected to network 520, defined by their respective information retrieval functions. Client 902 includes a client application 904, which is an information requesting or receiving application associated with client 902, and client 906 includes a client application 908, which is an information requesting or receiving application associated with client 906. Client applications 904, 908 can run either on clients 902, 906, respectively, or can run on another node and are then passed to the clients 902, 906. In one or more embodiments, the client applications 904, 908 are web browsers.

Servers 910-916 include a variety of processes, including operating systems, web server applications and application servers. The operating systems, which can also be called platforms, are the software programs that applications use to communicate with the physical parts of the servers 910-916. Examples of operating systems that can be used with the present invention include: Linux™, Sun Solaris™, Windows NT/2000™, Cobalt RaQ™, and Free BSD™, although any operating systems known or anticipated can be used.

The web server applications are software running on servers 910-916 that make it possible for the client browsers 904, 908 to download stored web pages. These applications also coordinate streaming audio, video, and secure e-commerce, and can be integrated with databases (as described below) for information retrieval. Examples of web server applications that can be used with the present invention include: Apache™, Microsoft's Internet Information Server (IIS)™, O'Reilly & Associates WebSite Pro™, Netscape's FastTrack Server™, and StarNine's WebSTAR™ (for Macintosh), although any operating systems known or anticipated can be used.

The application servers sit on top of the formatting and display languages (for example, HTML) such that a request from clients 902, 906 is generated and translated as a request to the databases. Upon receiving information from databases, the application servers will translate this information back to the formatting and display languages and sent a response back to the browser. In one or more embodiments, the application server software resides at the servers 910-916, although with cross-platform programming technology, software performing the same functions can reside at clients 902, 906 as well. In one or more embodiments, the application servers will insert strings of programming code into the formatting and display language, with client browsers 904, 908 employing interpreters (or a plug-ins) to translate back into the formatting and display language (for example, HTML) to display a page. Examples of application servers that can be used with the present invention include: Cactus™, Cold Fusion™, Cyberprise Server™, Ejipt™, Enterprise Application Server™, Netscape Application Server™, Oracle Application Server™, PowerTier for C++™, PowerTier for Enterprise Java Beans™, Secant Extreme™, Enterprise Server™, SilverStream™, WebEnterprise™, WebSpeed™, and WebSphere™ although any application servers known or anticipated can be used.

Taken together, the web servers and applications servers perform at least these functions: (i) providing an environment upon which server components can run; (ii) functioning as is a main program under which other components run as subroutines; (iii) providing services (for example, security related services, transaction related services), state management, and resources (for example, database connections); (iv) enabling communication with clients 902, 906.

For the convenience of condensing terminology, the aforementioned applications working, which work together on the servers 910-916 (or instead are processed at other nodes and passed to servers 910-916) are referred to as “application servers.” FIG. 16 illustrates applications servers (ASs) 922, 926, 930, 934 respectively can run on clients 910, 912, 914, 916. In operation, client browsers 904, 908 are used to issue requests for information, or queued to transmit information, over network 520. Requests and responses are handled by servers 910-916 via running of ASs 922, 926, 930, 934, which in turn transmit information over network 520 for display by browsers 904, 908.

In one or more embodiments, additional functions required of ASs 922, 926, 930, 934 will be to connect the web servers 910-916 to, for example, back-end data resources such as relational tables, flat files, e-mail messages, and directory servers. In exemplary embodiments, additional programs incorporated in ASs 922, 926, 930, 934 typically called “middleware,” database utilities, or database management systems (DMBS) can be used, among other known or anticipated database methods.

For example, the ASs 922, 926, 930, 934 can include their own internal DBMSs, or DBMSs of other nodes, or the DBMSs labeled database servers (DSs) 924, 928, 932, 936. The DBMS refers to computer software for storing, maintaining, and searching for data in a database. In the present invention, the DBMS can also utilize facilities for increasing reliability and performance, and integrity, such as indexes, logging, and record locking.

In one or more embodiments, the DBMS includes interfaces for searching for and locating particular data items from the database and for presenting the result of these queries to a search engine. A search engine as used herein searches the database in response to a user request, which can be initiated at client browser 902, 906, for example, or at server 922-924, for example, and returns a result to the user, for example in the form of a relational table viewable in browsers 904, 908. The DBMS can refer to any type of database, including a relational DBMS (RDBMS), LDAP™, VSAM™, IMS™, Active Directory Services™, message stores, to name a few.

In one or more embodiments, the DBMS is an RDBMS that uses relational database to retrieve information from the timing system 104 to obtain participant data including biometric data 120. In one or more embodiments, the relational database uses structured query language (SQL™), including SQL defined according to International Standards Organization (ISO) and American National Standards Institute (ANSI) standards, or follow these standards with additional language constructs. In one or more exemplary embodiments, ASs 922-924 are respectively connected to DSs 924-936 via an application programming interface (API), including for example the open database connectivity (ODBC™), Java database connectivity (JDBC™), APIs.

Any types of DBMS platforms can be used in the various systems and components of the systems described herein and methods thereof. Exemplary platforms that can be employed include Sun Microsystems' Java™, 2 Platform, Enterprise Edition (J2EE)™ that contains an Enterprise JavaBeans™. (EJB) server-side component architecture, and Microsoft's Windows™, Distributed interNet Applications Architecture (Windows DNA™), which contains the COM+™ server-side component architecture.

When describing elements or features and/or embodiments thereof, the articles “a”, “an”, “the”, and “said” are intended to mean that there are one or more of the elements or features. The terms “comprising”, “including”, and “having” are intended to be inclusive and mean that there may be additional elements or features beyond those specifically described.

Those skilled in the art will recognize that various changes can be made to the exemplary embodiments and implementations described above without departing from the scope of the disclosure. Accordingly, all matter contained in the above description or shown in the accompanying drawings should be interpreted as illustrative and not in a limiting sense.

It is further to be understood that the processes or steps described herein are not to be construed as necessarily requiring their performance in the particular order discussed or illustrated. It is also to be understood that additional or alternative processes or steps may be employed.

1. A system for reporting biometric data from a participant in an event comprising:
a sensor positioned proximate to a body of the participant for sensing a measured biometric parameter of the participant, the sensor including an interface transmitting the sensed measured biometric parameter;
an RFID tag associated with the participant and positioned proximate to the body of the participant, the RFID tag having a biometric data communication interface and a tag reader communication interface, the tag reader communication interface transmitting a tag identifier to a remote tag reader responsive to a tag read request therefrom; and
a biometric data module communicatively coupled to the sensor receiving the transmitted measured biometric parameter, having a memory storing the received measured biometric parameter, and having an output interface communicatively coupled to the biometric data communication interface of the RFID tag, the biometric data module transmitting the measured biometric parameter over the output interface;
wherein the RFID tag receives the transmitted measured biometric message parameter and transmits the received measured biometric parameter to the RFID tag reader along with the tag identifier responsive to the tag read request from the tag reader.

2. The system of claim 1, further comprising a tag reader associated a timing system used at the event in which the participant is involved, the tag reader transmitting a request to the RFID tag, wherein the RFID tag receives the request from the tag reader and transmits the tag identifier and the measured biometric parameter in response thereto, the tag reader receiving the tag identifier and the measured biometric parameter as transmitted by the RFID tag and transmitting the received tag identifier and the measured biometric parameter over a timing system communication interface to a remote system.

3. The system of claim 2, further comprising a timing system having a first communication interface communicatively coupled to the timing system communication interface of the tag reader, the timing system receiving the tag identifier and the measured biometric parameter, storing each, and providing the tag identifier or a participant identifier associated with the tag identifier and the received measured biometric parameter over a second communication interface.

4. The system of claim 3, further comprising a biometric module receiving the tag identifier and the measured biometric parameter from the second interface of the timing system, identifying the participant associated with the tag identifier, receiving the measured biometric parameter, comparing the received measured biometric parameter to a predefined biometric parameter value and determining a biometric status of the identified participant as a result of the comparing process.

5. The system of claim 3 wherein the biometric module includes an output interface and the biometric module transmits the determined biometric status of the participant over the output interface to one or more predefined biometric reporting devices.

6. The system of claim 5 wherein the biometric module formats the determined biometric status for transmitting over the output interface thereof for displaying the determined biometric status on a visual display along with the identification of the participant.

7. The system of claim 4 wherein the biometric module is a module of at least one of the timing system, a standalone biometric system, a module of a remote participant management system, and a module of a medical monitoring and reporting system.

8. The system of claim 4 wherein the timing system determines a time associated with each received measured biometric parameter and transmits the determined time with the measured biometric parameter, and wherein the biometric module receives the determined time, and wherein the determining of the biometric status is a function of the determined time.

9. The system of claim 4 wherein the timing system identifies a location of the participant associated with the received measured biometric parameter and transmits the identified location with the measured biometric parameter, and wherein the biometric module receives the identified location and wherein the determining of the biometric status is a function of the identified location.

10. The system of claim 9, further comprising a participant location system identifying a location of the participant, the participant location system providing the identified location to the timing system.

11. The system of claim 5 wherein the predefined biometric parameter value is a range of values or a threshold value for the biometric value, wherein the determining of the biometric status by the biometric module includes identifying the existence of a participant alert condition, the biometric module further generating an alert message over the output interface of the biometric module to at least one of the biometric reporting devices including a notification of the identified participant alert condition.

12. The system of claim 6, further comprising a participant reporting device positioned proximate to the participant during the event, the participant reporting device including an input interface receiving the determined biometric status as transmitted from the biometric module and an output interface providing an indication to the participate of the received determined biometric status of the participant.

13. The system of claim 4, further comprising a participant image capture system capturing an image of the participant during the event and providing the captured image to the timing system, wherein the captured image is associated with the measured biometric parameter of the participant.

14. The system of claim 2 wherein the received measured biometric parameter is a first value for the measured biometric parameter and wherein the biometric data module, and the biometric data module received a second value for the measured biometric parameter at a later time period, and wherein the tag reader is a first tag reader positioned at a first location along a route of the event, further comprising a second tag reader positioned at a second location along the route of the event that is spaced apart from the first location and being communicatively coupled to the timing system, the second tag reader transmitting a second request to the RFID tag, wherein the RFID tag receives the second request from the second tag reader and transmits the tag identifier and the second value for the measured biometric parameter in response thereto, the second tag reader receiving the tag identifier and the second value for the measured biometric parameter as transmitted by the RFID tag and transmits the received tag identifier and the second value for the measured biometric parameter over the timing system communication interface to the remote system.

15. The system of claim 14, further comprising a biometric module receiving the tag identifier and the first value for the measured biometric parameter and the second value for the measured biometric parameter, identifying the participant associated with the tag identifier, comparing the received first value and second value of the measured biometric parameter together or to a predefined biometric parameter value and determining a biometric status of the identified participant as a result of the comparing process.

16. The system of claim 1 wherein the biometric data module is configured as an interconnected module of the RFID tag.

17. The system of claim 1 wherein the sensor senses a measured biometric parameter of the participant selected from the group of parameters consisting of a heart rate, a pulse rate, a blood pressure, a core body temp, a sweat level or rate, a skin oxidation, a breathing rate, and a chemical content of a breath.

18. A system for reporting biometric data from a participant in an event comprising:
a sensor positioned proximate to a body of the participant for sensing a measured biometric parameter of the participant, the sensor including an interface transmitting the sensed measured biometric parameter;
an RFID tag associated with the participant and positioned proximate to the body of the participant, the RFID tag having a biometric data communication interface and a tag reader communication interface, the tag reader communication interface transmitting a tag identifier to a remote tag reader responsive to a tag read request therefrom; and
a biometric data module communicatively coupled to the sensor receiving the transmitted measured biometric parameter, storing the received measured biometric parameter in a memory of the biometric data module, and having an output interface transmitting the stored measured biometric parameter over the output interface;
wherein the RFID tag receives the transmitted measured biometric message parameter and transmits the received measured biometric parameter to the RFID tag reader along with the tag identifier responsive to the tag read request from the tag reader.

19. The system of claim 18 wherein the output interface of the biometric module is a wireless interface selected from the group consisting of a Wi-Fi interface, a Bluetooth interface, a mobile telephone interface, and a wireless interface.

20. The system of claim 19 wherein the output interface of the biometric data module is communicatively coupled via local wiring or a wireless communication link to a biometric input interface of the RFID tag.

21. The system of claim 19 wherein the biometric data module is further configured to transmit the measured biometric parameter directly to a remote wireless communication system other than the RFID tag.

22. The system of claim 18, further comprising a timing system having a first communication interface communicatively coupled to the RFID tag for receiving the transmitted tag identifier and communicatively coupled to the biometric data module for receiving the measured biometric parameter, and a second communication interface providing the received tag identifier or a participant identifier associated with the tag identifier and the received measured biometric parameter.

23. The system of claim 22, further comprising a biometric module receiving the tag identifier and the measured biometric parameter from the second interface of the timing system, identifying the participant associated with the tag identifier, receiving the measured biometric parameter, comparing the received measured biometric parameter to a predefined biometric parameter value and determining a biometric status of the identified participant as a result of the comparing process.

24. The system of claim 23 wherein the biometric status is an alert condition and wherein the system generates an alert message over the output interface for communication to a participant biometric status reporting device for presenting an alert indicator to the participant.

25. The system of claim 22 wherein the biometric module includes an output interface and the biometric module transmits the determined biometric status of the participant over the output interface to one or more predefined biometric reporting devices.

26. The system of claim 25 wherein the biometric module formats the determined biometric status for transmitting over the output interface thereof for displaying the determined biometric status on a visual display along with the identification of the participant.

27-39. (canceled)
